SEQUENCE LISTING (18 sequences)

SEQ ID NO: 1 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 2 - protein, 18 aa, Artificial Sequence
  K1 site
PEFYYRPPRPPKGACKVP

SEQ ID NO: 3 - protein, 16 aa, Artificial Sequence
  K2 site
DVSGSRPAAPLIGAPA

SEQ ID NO: 4 - protein, 16 aa, Artificial Sequence
  K3 site
GSVEQTPKKPGLRRRQ

SEQ ID NO: 5 - protein, 59 aa, Artificial Sequence
  Brk SH3
KYVGLWDFKSRTDEELSFRAGDVFHVARKEEQWWWATLLDEAGGAVAQGYVPHNYLAER

SEQ ID NO: 6 - protein, 56 aa, Artificial Sequence
  Src SH3
TFVALYDYESRTETDLSFKKGERLQIVNNTEGDWWLAHSLTTGQTGYIPSNYVAPS

SEQ ID NO: 7 - protein, 4 aa, Artificial Sequence
  Synthetic sequence
PPPP

SEQ ID NO: 8 - protein, 4 aa, Artificial Sequence
  Synthetic sequence
AAAA

SEQ ID NO: 9 - protein, 4 aa, Artificial Sequence
  Synthetic sequence
PKKP

SEQ ID NO: 10 - DNA, 26 bp, Artificial Sequence
  Forward primer
ggcctcgagctagctctcctgcgccg

SEQ ID NO: 11 - DNA, 65 bp, Artificial Sequence
  Reverse primer
ggggtctagagccaccatggactacaaggacgacgatgacaagcgcaagtggaatttcgattttc

SEQ ID NO: 12 - protein, 134 aa, Artificial Sequence
  Alt-Brk
MVSRDQAHLGPKYVGLWDFKSRTDEELSFRAGDVFHVARKEEQWWWATLLDEAGGAVAQGYVPHNYLAERETVESEPAGHAGCAALQDLAACRGPAAPERGGVLPQPARACELPQGPEPVPRPAAGRALPEARA

SEQ ID NO: 13 - DNA, 24 bp, Artificial Sequence
  Actin forward primer
aaaatctggcaccacaccttctac

SEQ ID NO: 14 - DNA, 22 bp, Artificial Sequence
  Actin reverse primer
tagcacagcctggatagcaacg

SEQ ID NO: 15 - DNA, 20 bp, Artificial Sequence
  Brk forward primer
ccaagtatgtgggcctctgg

SEQ ID NO: 16 - DNA, 20 bp, Artificial Sequence
  Brk reverse primer
aaagaaccacggttccgact

SEQ ID NO: 17 - DNA, 20 bp, Artificial Sequence
  Alt-Brk forward primer
gacggtggagtcggaacctg

SEQ ID NO: 18 - DNA, 20 bp, Artificial Sequence
  Alt-Brk reverse primer
tagttcacaagctcgggcag

(19) United States
(12) Patent Application Publication
(10) Pub. No.: US 2019/0369104 A1
(43) Pub. Date: Dec. 5, 2019

(54) P27 TYROSINE PHOSPHORYLATION AS A MARKER OF CDK4 ACTIVITY AND METHODS OF USE THEREOF

(71) Applicant: THE RESEARCH FOUNDATION FOR THE STATE UNIVERSITY OF NEW YORK, Albany, NY (US)

(72) Inventor: Stacy W. Blain, Brooklyn, NY (US)

(21) Appl. No.: 16/075,900

(22) PCT Filed: Feb. 23, 2017
(86) PCT No.: PCT/US2017/019184

§ 371 Date: Aug. 6, 2018

Related U.S. Application Data
(60) Provisional application No. 62/298,584, filed on Feb. 23, 2016.

(51) Int. Cl. G01N 33/574; A61P 35/00; A61K 31/519; A61K 45/06

(52) U.S. Cl. CPC G01N 33/57496; A61P 35/00; G01N 33/57415; A61K 45/06; A61K 31/519

(57) ABSTRACT

Compositions and methods for the treatment of malignancy are disclosed. Specifically, the disclosure provides a method for treating cancer comprises assessing tyrosine 88 (Y88) phosphorylation (pY88) levels in p27 in a biological sample comprising cancer cells from a subject, and stratifying pY88 phosphorylation levels as 0, 1, 2 or 3 as compared to pY88 phosphorylation levels observed in control tissues; where a level of 0 indicates no detectable sensitivity to cyclin-dependent kinase 4 (cd4k) inhibition; a level of 1, low or no detectable sensitivity; and a level of 2 or 3, indicates detectable sensitivity to cdk4 inhibition. Further provided is a kit for practicing the method.

This application is a § 371 application of PCT/US2017/019194, filed Feb. 23, 2017 which claims priority under 35 U.S.C. § 119(e) to U.S. Provisional Patent Application No. 62/298,584, filed Feb. 23, 2016. The foregoing application is incorporated by reference herein.

### FIELD OF THE INVENTION

This invention relates to the fields of cancer and cancer treatment and management. More specifically the invention provides diagnostic methods for identifying those subjects most likely to benefit from cdk4/cdk2 modulation and methods of treatment for subjects so identified.

### BACKGROUND OF THE INVENTION

Several publications and patent documents are cited throughout the specification in order to describe the state of the art to which this invention pertains. Each of these citations is incorporated herein by reference as though set forth in full.

Breast cancer is the second leading cause of cancer mortality in women in the USA, with ^{˜}40,000 deaths per year. The American Cancer Society estimates 232,000 new cases of invasive breast cancer and ^{˜}60,000 cases of ductal carcinoma in situ will occur this year. Advances in molecular diagnostics have revealed that breast cancer is not a single disease entity; rather, it is a diverse disease with extensive intertumoral and intratumoral heterogeneity (i.e., subclones of cells with differing genetic, epigenetic, and/or phenotypic characteristics). This heterogeneity has significant clinical and therapeutic consequences in terms of patient prognosis and response to hormonal and targeted therapies, in addition to response to chemotherapies.

Growing knowledge of the molecular underpinnings comprising the etiology of cancer has driven the field of personalized or “precision” medicine to identify specific tumor characteristics and exploit these features by developing targeted therapies against these entities. The ability to predict an individual's response to a specific therapy is the ultimate goal in modern precision medicine. Several targeted cancer therapies are currently utilized in standard oncological care as a result of the more detailed genetic and clinical understanding of individual tumor characteristics. The therapeutic use of molecular biomarkers with predictive clinical and pharmacological relevance relies on accurately detecting and/or quantifying these biomarkers to direct the safe and effective treatment of targeted therapies.

Clearly, there is an urgent need to provide sensitive diagnostic assays and treatment regimens designed to target the particular type of cancer cells present in the tumor. It is an object of the invention to provide such assays and treatment protocols.

### SUMMARY OF THE INVENTION

In accordance with the present invention, a method for treating cancer in a subject is provided. An exemplary method comprises assessing pY88 phosphorylation levels in p27 in a biological sample comprising cancer cells from a subject and stratifying pY88 phosphorylation levels as 0, 1, 2 or 3 as compared to pY88 phosphorylation levels observed in control tissues, where a level of 0 indicates no detectable sensitivity to cdk4 inhibition, a level of 1, low or no detectable sensitivity and a 2 or 3 indicates detectable sensitivity to cdk4 inhibition.

Subjects identified as having tumors sensitive to cdk4 inhibition are then treated with a therapeutically effective amount of at least one cdk4 inhibitor for the alleviation of cancer burden or symptoms. Cdk4 inhibitors may be administered alone or in combination with other anti-cancer agents. Cancers to be treated in accordance with the invention include, without limitation, cancers of the breast, brain, thyroid, prostate, colorectum, pancreas, cervix, stomach, endometrium, liver, bladder, ovary, testis, head, neck, skin, mesothelial lining, white blood cell, esophagus, muscle, connective tissue, lung, adrenal gland, thyroid, kidney, bone, and stomach. In a preferred embodiment, the test and treat method of the invention is used for the treatment of breast cancer. While the invention encompasses treatment of a variety of mammals, preferably, the mammal is a human.

Cdk inhibitors that can be employed in the practice of the invention are described herein and include the cdk inhibitors listed in Table 2. In certain embodiments, cdk4 and cdk2 inhibitors are administered in combination. This combination may or may not include additional anti-cancer agents. A preferred therapeutic for use in the invention, includes a mimetic of p27 or an Alt-Brk mimetic. In a particularly preferred embodiment, the cdk4 inhibitor is Palbociclib. In a further preferred embodiment an Alt-Brk mimetic is also administered which acts synergistically with Palbociclib to kill cancer cells.

The present invention also provides a method for assessing efficacy of inhibition of cdk4 activity in cancer treatment comprising comparing pY88 phosphorylation levels in p27 in biological samples comprising cancer cells from said subject before and after treatment with an anti-cancer agent, wherein said anti-cancer agent comprises one or more cdk inhibitors samples and stratifying levels as 0, 1, 2, or 3, wherein a reduction in Y88 phosphorylation level is correlated with efficacy of cdk4 inhibition and reduced cancer cell proliferation and an increase of Y88 phosphorylation level is correlated with reduced or loss of efficacy of cdk4 inhibitor therapy.

Kits for practicing the methods disclosed herein are also provided.

### BRIEF DESCRIPTION OF THE DRAWINGS

FIGS. 1A-1D: Brk binds to p27 with high affinity in vitro. (FIG. 1A) p27 sequence highlighting the proline tracts of the three putative SH3 domain recruitment sites (PxxP): K1 (90-96), K2 (114-117) and K3 (188-195) (SEQ ID NO: 1). Phage-ELISA-analysis of SFK SH3 interactions with p27 (FIG. 1B) or p27's PxxP motifs (FIG. 1C). Data shown is the mean of three independent experiments±standard deviation after normalization and subtraction of the background binding to GST. (FIG. 1C) Recombinantly produced GST-K1, -K2, -K3 (SEQ ID NOS: 2, 3 and 4, respectively) or GST was immobilized in 96-well plates and analyzed for binding of the phages with the Brk-, Frk-, Yes, or Abl-SH3 domain. An alternative splice variant of Brk, Alt Brk, which lacks expression of exon 2 and encodes a shorter, 15-kDa protein is shown in FIG. 1D. Alignment of 3D structures of Brk (SEQ ID NO: 5) and Src SH3 (SEQ ID NO: 6) domains, derived from the PDB+jFATCAT rigid databases. Brk SH3 in orange, Src SH3 in blue is shown. This Alt Brk shares the N-terminal SH3 domain with Brk and has a unique proline-rich carboxy terminus but lacks the catalytically active SH1 kinase domain.

FIG. 2. MCF7 cells that overexpressed WT Brk, or a catalytically inactive version (KM) were generated. When p27 was immunoprecipitated from these cells, immunoblot analysis with anti pY88, pY74 or p27 antibodies was performed, demonstrating that increased pY88 was detected in the cells that overexpressed Brk. When cdk4 was immunoprecipitated from these cells, and used in in vitro RB kinase assays with recombinant RB, increased cdk4 kinase activity was detected from the cells that expressed WT Brk. The cells that expressed WT Brk proliferated faster than the mock expressing cells. Increased Brk, increased pY88, increased cdk4 kinase activity and increased PD resistance. The MCF7-Wt Brk expressing cells had an IC₅₀ value of ˜v600 nM PD.

FIG. 3. Breast cancer cell panel showing Palbociclib sensitivity. IC50 values in nM plotted (from Finn, R. S., et al., Breast Cancer Res, 2009. 11(5): p. R77).

FIGS. 4A-4C. Paraffin-embedded cell block material from high responders MCF7 (FIG. 4A), Moderate MDA MB 231 (FIG. 4B), or non-responders HCC1954 (FIG. 4C) with p27 (brown) and pY88 (red) antibodies and showing low (FIG. 3A), moderate (FIG. 3B) and high (FIG. 4C) pY88 levels.

FIGS. 5A-5C. p27 Y88 serves as a cdk4 biomarker. (FIG. 5A) Asynchronous MCF7, MDA MB231 or HCC1954 cells were treated with DMSO or MCF7 cells were treated with 400 nM PD. Cells blocks were made after harvesting and fixing the cells with 10% Formalin. Immunohistochemistry was performed staining the slides with p27 (Brown) and pY88 (Pink). (FIG. 5B) Needle biopsies from normal mammary epithelium or ER/PR+ HER2− breast cancer patients were stained with p27 (brown) and pY88 (pink) antibodies. Patients were categorized on the % pY88+ cells (green, yellow, brown) and whether the staining was pY88 strong (purple, grey, red). Staining was analyzed blindly by 2 independent pathologists. (FIG. 5C) Tables summarizing the staining results are shown.

FIG. 6. Material discarded from lumpectomy or mastectomy from ER/PR+, Her2− patients at University Hospital was grown in explant culture for 48 h. in DMSO (green), high non-physiological Palbociclib (red), or a physiological concentration of Palbociclib (purple). After 48 h. material was fixed, paraffin embedded and stained for Ki67, as a marker of proliferation. The high concentration of drug (purple) was an internal control that proliferation could be inhibited. Each patient had an inherent different proliferation rate as measured by different Ki67 levels in the untreated sample (DMSO). Palbociclib response was measured as a decrease in Ki67 in the presence of the physiological concentration of drug (red). Patients 1 and 3 responded. Each data point is the average of 4 samples (2 independent explant samples, and 2 independent immunohistochemistry stainings). Each sample was read blindly by two pathologists.

### DETAILED DESCRIPTION OF THE INVENTION

Cyclin D-cdk4 (DK4) provides an ideal therapeutic target because it drives cancer proliferation in a majority of human tumors, including ER/PR+, Her2− breast cancer. Cyclin D and cdk4 are over-expressed in a variety of tumors, but their levels are not accurate indicators of oncogenic activity because an accessory factor, e.g., p27Kip1, is required to assemble this unstable dimer into a ternary complex. Additionally, tyrosine (Y) phosphorylation of p27 (pY88) is required to activate cdk4, acting as an ON/OFF “switch.” The present invention identifies an SH3 recruitment domain within p27 that modulates pY88, thereby modulating cdk4 activity. Using an SH3:PxxP interaction screen, a Brk (Breast Tumor Kinase) was identified as a high-affinity p27 kinase. Further mutational studies of p27 enabled the present inventors to identify the SH3 recruitment domain required to permit Y phosphorylation in vitro and in vivo. Modulation of Brk in breast cancer cells modulates pY88 and increases resistance to the cdk4 inhibitor, PD0332991 (Palbociclib). An alternatively-spliced form of Brk (Alt Brk), which contains its SH3 domain, blocks pY88 and acts as an endogenous cdk4 inhibitor, identifying a potentially targetable regulatory region within p27. Brk is overexpressed in 60% of breast carcinomas, suggesting that this facilitates cell cycle progression by modulating cdk4 through p27 Y phosphorylation. p27 has been considered a tumor suppressor, but the data herein strengthen the idea that it should also be considered an oncogene, responsible for cyclin D-cdk4 activity. Phosphorylation of Tyr-88/Tyr-89 in the 3¹⁰ helix of p27 reduces its cyclin-dependent kinase (CDK) inhibitory activity. This modification does not affect the interaction of p27 with cyclin-CDK complexes but does interfere with van der Waals and hydrogen bond contacts between p27 and amino acids in the catalytic cleft of the CDK, allowing the C-terminus of p27 to exit the catalytic site. The cyclin D-cdk4 complex is held together by p27, but p27 Y phosphorylation acts as a “switch” opening or closing the complex to permit catalytic activity. Thus, it had been suggested that phosphorylation of this site could switch the tumor-suppressive CDK inhibitory activity to an oncogenic activity.

Currently, several cdk4 inhibition therapies (cdk4i) have been developed and are in various stages of FDA approval. Unfortunately, a biomarker to pinpoint tumors and patients that would be responsive to cdk4 inhibition therapy does not exist. As described above, a tyrosine phosphorylation on residue Y88 and or Y89 of p27 is required to convert this ternary complex from an inhibited complex to an active complex. Accordingly, pY-associated p27 identified herein is advantageously used as a marker for cdk4 activity. Thus, the present invention encompasses compositions and methods using pY as a marker in human patient material to determine whether a particular tumor has the range of cdk4 activity that the present inventors have identified as responsive to treatment with cdk4 inhibitors. We have developed a phosphospecific antibody for pY p27 and shown that it recognized pY in paraffin embedded archival human breast cancer material (ER/PR+/Her2−). With 100% penetrance, the antibody did not stain benign tissue obtained from human mammary reduction surgery. Approximately 75% of the ER/PR+/Her-2 breast cancer samples analyzed stained positive for pY (47% high staining, 25% moderate staining).

The diagnostic test and treat method of the invention enables the clinician to more accurately identify those patients that will benefit from cdk inhibitor therapy. Patients identified as having cdk4 activity at levels amenable to therapy are then treated with cdk4 inhibitor therapy, alone or in combination with other chemotherapeutic or anti-proliferative agents.

Notably, pY can also be used as a surrogate marker to assess efficacy of anti-cancer treatment in such patients. For example, if the cdk4i therapy is effective and cyclin D-cd4 activity is off, pY will not be phosphorylated. If the cdk4i therapy ceases to be effective, thereby restoring cyclinD-cdk4 activity, pY will again be present.

### I. Definitions

A “therapeutically effective amount” of a compound or a pharmaceutical composition refers to an amount sufficient to modulate tumor growth or metastasis in an animal, especially a human, including without limitation decreasing tumor growth or size or preventing formation of tumor growth in an animal lacking any tumor formation prior to administration, i.e., prophylactic administration.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on human immunoglobulin sequences.

The “anti-cancer agent” in this specification refers to a chemical substance having cytotoxic or anti-proliferative effects on cancer cells.

The “chemotherapy” in this specification is therapy for a malignant tumor in the living body by administering the anti-cancer agent into the living body.

Chemotherapy for breast cancer includes, for example, CMF therapy (therapy by administering a combination of 3 agents, those are, cyclophosphamide, methotrexate and fluorouracil), therapy using taxane-based anticancer agents such as docetaxel, paclitaxel etc., CE therapy (therapy by administering a combination of 2 agents, that is, cyclophosphamide and epirubicin), AC therapy (therapy by administering 2 agents, that is, doxorubicin and cyclophosphamide), CAF therapy (therapy by administering a combination of 3 agents, that is, fluorouracil, doxorubicin and cyclophosphamide), FEC therapy (therapy by administering a combination of 3 agents, that is, fluorouracil, epirubicin and cyclophosphamide), therapy by administering a combination of 2 agents, that is, trastuzumab and paclitaxel, and therapy using capecitabine. Other treatment modalities include use of herceptin, alone and in combination with other anti-cancer agents. Notably, cdk4 inhibition therapy can also be used to advantage in certain breast cancer patients. Sensitivity to cdk4 directed chemotherapy can be determined by comparing the level of pY88 phosphorylation in the patient prior to treatment, as Y88 serves as predictor for responsiveness.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleosidephosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting p27 mRNA may be between 15-35 nucleotides in length, and more typically about 21 nucleotides in length.

As used herein, “mimetic of p27” can refer to a peptide variant, a fragment thereof, organic compound or small molecule which has the same function/structure-activity of the cdk4 modulating domains within p27. Alt-Brk, the alternative transcript of Brk encodes a 134 amino acid protein, which shares the first 77 amino acid residues including the SH3 domain with full length Brk. Mimetics of BRK-alt (or the SH3 domain thereof) are also provided herein. When the “mimetic” is a peptide variant, the length of its amino acid sequence is generally similar to that of the K1-containing peptide, an SH3-binding peptide in p27 or an SH3 containing peptide in Alt-Brk. Alternatively, such “mimetic” can be the peptide variants having a shorter length of the amino acid sequence.

Suitable mimetics or analogues can be generated by modeling techniques generally known in the art. This includes the design of “mimetics” which involves the study of the functional interactions and the design of compounds which contain functional groups arranged in such a manner that they could reproduce those interactions.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “oligonucleotide” or “oligo” as used herein means a short sequence of DNA or DNA derivatives typically 8 to 35 nucleotides in length, primers, or probes. An oligonucleotide can be derived synthetically, by cloning or by amplification. An oligo is defined as a nucleic acid molecule comprised of two or more ribo- or deoxyribonucleotides, preferably more than three. The exact size of the oligonucleotide will depend on various factors and on the particular application and use of the oligonucleotide. The term “derivative” is intended to include any of the above described variants when comprising an additional chemical moiety not normally a part of these molecules. These chemical moieties can have varying purposes including, improving solubility, absorption, biological half life, decreasing toxicity and eliminating or decreasing undesirable side effects.

“Concurrently” means (1) simultaneously in time, or (2) at different times during the course of a common treatment schedule.

“Sequentially” refers to the administration of one active agent used in the method followed by administration of another active agent. After administration of one active agent, the next active agent can be administered substantially immediately after the first, or the next active agent can be administered after an effective time period after the first active agent; the effective time period is the amount of time given for realization of maximum benefit from the administration of the first active agent.

The term “subject” refers to mammalian subjects, including but not limited to humans, dogs, livestock, horses, cats, rabbits and the like. Preferably, the subject is a human subject.

A “cdk4 inhibitor” or “cdki” is an agent (e.g., nucleic acid, protein/peptide, small molecule) that disrupts or interferes with cdk4 kinase activity. Such inhibitors include, without limitation, agents listed in Table 2, Palbociclib, abemaciclib, and ribociclib. Also see U.S. Pat. Nos. 8,566,072 and 6,962,792.

### II. Therapy for the Treatment of Cancer

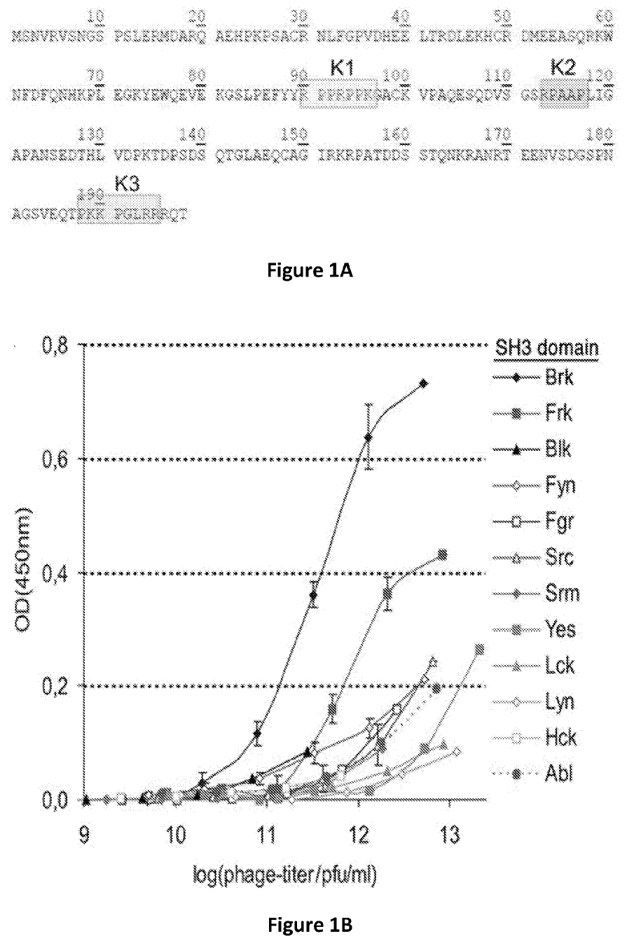

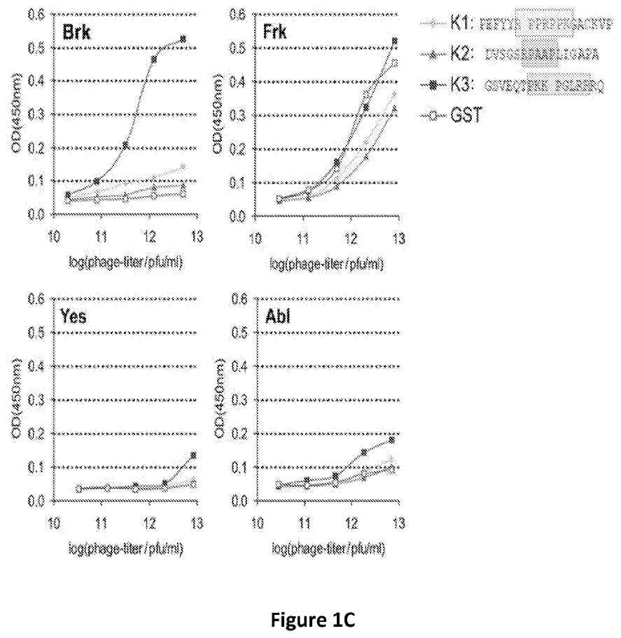

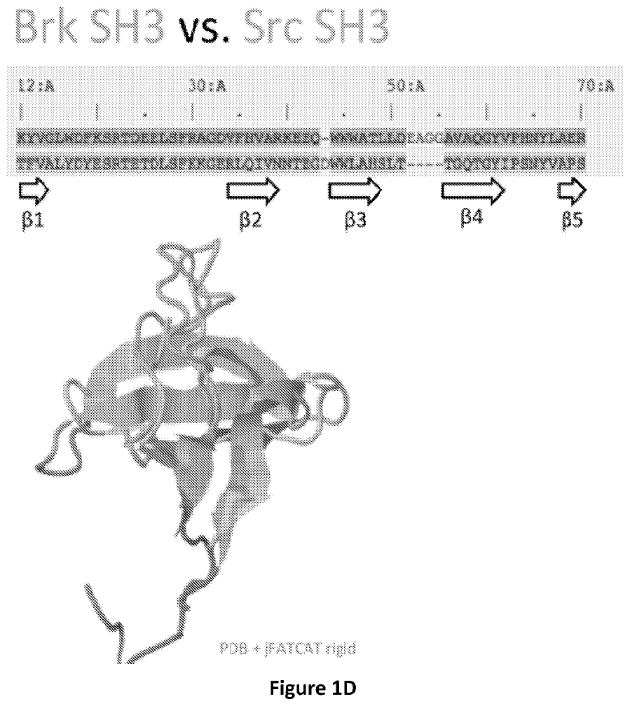

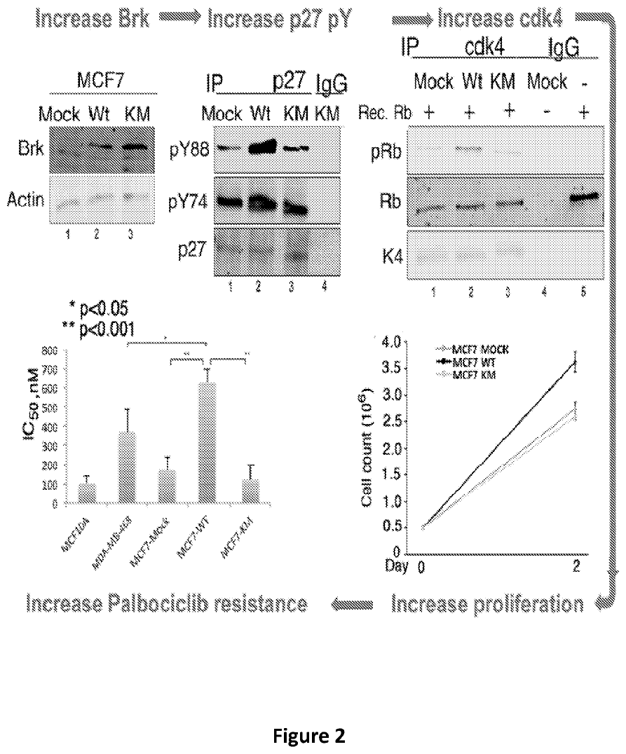

The present invention also provides pharmaceutical compositions comprising at least one agent, wherein the at least one agent is a compound which interferes with the interaction between p27Kip1 and Brk and inhibits the phosphorylation event that turns p27 “on” in a pharmaceutically acceptable carrier. Preferred agents for use in the invention include small molecules, cdk4 inhibitors such as those listed in Table 2, mimetics based on the sequences provided in FIG. 1, and siRNA. Such a pharmaceutical composition may be administered, in a therapeutically effective amount, to a patient in need of cancer treatment.

The mimetics/siRNA/inhibitors of the present invention may be used in a variety of treatment regimens for the treatment of malignant disease. Cancers that may be treated using the present protocol include, but are not limited to: cancers of the breast, brain, thyroid, prostate, colorectum, pancreas, cervix, stomach, endometrium, liver, bladder, ovary, testis, head, neck, skin (including melanoma and basal carcinoma), mesothelial lining, white blood cell (including lymphoma and leukemia) esophagus, muscle, connective tissue, lung (including small-cell lung carcinoma and non-small-cell carcinoma), adrenal gland, thyroid, kidney, or bone; glioblastoma, mesothelioma, renal cell carcinoma, gastric carcinoma, sarcoma, choriocarcinoma, cutaneous basocellular carcinoma, and testicular seminoma.

It should be understood that treatment may occur prior to tumor resection or following tumor resection for example.

### III. Combination Therapies for the Treatment of Cancer

In accordance with the present invention, it has also been discovered that the combination of the agents and small molecules/mimetics/siRNA described herein with certain known chemotherapeutically effective agents act synergistically to suppress tumor growth. Accordingly, the present invention provides a pharmaceutical composition for the treatment of cancer in a patient comprising at least one agent that interferes with specific tyrosine (Y) phosphorylation, thereby maintaining p27 in the “off” position and at least one chemotherapeutic agent in a pharmaceutically acceptable carrier. Also provided is a method for treating cancer in a patient by administering an effective amount of at least one Y88 phosphorylation inhibiting agent. Such agent can be used alone or in combination with at least one other anti-cancer agent. Suitable agents include, but are not limited to, Palbociclib, abemaciclib, ribociclib, paclitaxel (Taxol®), cisplatin, docetaxel, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives. Such agents can be administered simultaneously or sequentially.

### IV. Administration of Pharmaceutical Compositions and Compounds

The pharmaceutical compositions of the present invention can be administered by any suitable route, for example, by injection, by oral, pulmonary, nasal or other methods of administration. In general, pharmaceutical compositions of the present invention, comprise, among other things, pharmaceutically acceptable diluents, preservatives, solubilizers, emulsifiers, adjuvants and/or carriers. In certain instances, the carriers are nanoparticles. Such compositions can also include diluents of various buffer content (e.g., Tris-HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. The pharmaceutical composition of the present invention can be prepared, for example, in liquid form, or can be in dried powder form (e.g., lyophilized). Particular methods of administering pharmaceutical compositions are described hereinabove.

In yet another embodiment, the pharmaceutical compositions of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity of the site of inappropriate immune activation or a tumor. Other controlled release systems are discussed in the review by Langer (Science (1990) 249:1527-1533).

### V. Kits and Articles of Manufacture

Any of the aforementioned products can be incorporated into a kit which can comprise one or more of antibodies immunospecific for Y88 and Y89 in phosphorylated and non phosphorylated forms, Y88 and Y89 antigens, said antibodies optionally being detectably labeled with a non naturally occurring detectable label, or optionally affixed and immobilized to a solid support, an oligonucleotide which is suitable for amplification or specific hybridization with p27 encoding nucleic acids, a non naturally occurring polypeptide mimetics of p27 and Alt-brk, a pharmaceutically acceptable carrier, instructions for use, a container, a vessel for administration, an assay substrate, a cdk inhibitor, an anti cancer agent, or any combination thereof.

The following materials and methods are provided to facilitate the practice of the present invention.

### Antibodies.

Mouse Anti-p27(Kip1), BD Biosciences 610242. Cdk4 (DCS-35), p27 (N-20), C-terminal Brk (C-18), Brk (D-6), N-terminal Brk (N-20), c-Src (SC-18), Cyclin D1 (H 295), ARHGDIA (A-20), Santa Cruz Biotechnology. Phosphotyrosine (P-Tyr-100), Cell Signaling Technology. Cdk4 (C-term, Cat. No. AP7520b), Abgent. GST (PRB-112C), Covance. Flag (F3165), Actin (A2066), Sigma Aldrich. PhosphoBrk (Tyr342), EMD Millipore. pY74, Y88 and Y89 phospho-specific antibodies were generated by immunization of rabbits with phosphor-specific p27 peptides (Invitrogen). Negative- and positive-affinity chromatography with non-phosphorylated and phosphorylated peptides respectively, were performed to purify the antibodies. The antibodies are specific only for Y88, Y89, Y74 phosphorylation respectively.

### Enzymes.

Gst-PTK6/Brk, GST-Src (SignalChem), His-Abl (New England Biolabs), His-PTK6/Brk, His-Src (Invitrogen) were used according to manufacturer's specifications. Enzymes had approximately equivalent specific activities.

### Phage-ELISA.

Phage supernatants were generated and binding of SH3-phages to recombinantly produced His-tagged-p27 or GST-PxxP-peptides were analyzed as described (Asbach B. et al., (2012) PLoS One 6: e38540).

### Construction of Mutants and Peptides.

Oligonucleotides encoding the PxxP-peptides K1, K2 and K3 were annealed and directly ligated into pGEX-KG expression vector for production of N-terminally GST-tagged peptides. GST, GST-Brk SH3, GST-Brk SH2 expressing plasmids were described (Vasioukhin V. et al; (1997) Proc. Natl. Acad. Sci. 94: 14477-82). E. coli BL21 cells transformed with these plasmids were grown in LB-ampicillin until an OD of 0.6 was reached and protein production was induced by addition of 1 mM IPTG. After 2 hours, cells were harvested by centrifugation. Cell lysis and protein purification on GST-sepharose was carried out according to the GST-protein purification manual (GE Healthcare). Protein was eluted with an excess of glutathione and dialysed against PBS for further use. Purified, C-terminal histidine-tagged or N-terminal Flag tagged p27's were generated from E. coli as described previously (James M. K. et al. (2008) Mol. Cell. Biol. 1: 498-510). Human p27 cDNA was used as a template in PCR-mutagenesis with oligonucleotides carrying the point mutations: PPPP (SEQ ID NO: 7) 91,92,94,95AAAA (SEQ ID NO: 8) (ΔK1); PKKP (SEQ ID NO: 9) 188,189,190,191 AAAA (SEQ ID NO:8) (ΔK3); or PPPP (SEQ ID NO: 7) 91,92,94,95AAAA (SEQ ID NO: 8) and PKKP (SEQ ID NO: 9) 188,189,190,191 AAAA (SEQ ID NO: 8 (ΔK1/K3).

Oligonucleotides Used to Generate 58-106 were:

Forward primer
(SEQ ID NO: 10)
5′-GGCCTCGAGCTAGCTCTCCTGCGCCG-3′
Reverse primer
(SEQ ID NO: 11)
5′GGGGTCTAGAGCCACCATGGACTACAAGGACGACGATGACAAGCGC
AAGTGGA ATTTCGATTTTC-3′

The PCR fragments were ligated to the T7pGEMEX human His-p27 or T7pGEMEX humanFlag-p27 plasmid for expression in E. coli. Mutants Y74F, Y88F, and Y88/89F were previously described (See James et al., supra). Flag-tagged p27 mutants were purified by Flag-immunoprecipitation with Flag antibody (M-2, Sigma F-18C9) and eluted with Flag peptide (Sigma F-4799) according to manufacturer's protocol. His-tagged p27 mutants were purified by FPLC via his-trap affinity chromatography (His-Trap HP, GE Healthcare 71-5247-01). The affinity column was stripped according to manufacturer's protocol, then washed with 5 column volumes of 100 mM CoCl₂. The crude material was applied with a loading buffer consisting of 6 M urea, 500 mM NaCl, 50 mM Tris-HCl, pH 7.5 and 20% glycerol. The material was washed with 500 mM NaCl, 50 mM Tris-HCl, pH 7.5 and 10% glycerol. The purified material was eluted with 500 mM imidazole, 20 mM Hepes pH 7.4 and 1 M KCl. The protein was then dialyzed overnight in a solution of 25 mM Hepes pH 7.7, 150 mM NaCl, 5 mM MgCl2 and 0.05% NP40. All purified proteins were analyzed by Coomassie and immunoblot analysis. The p27, ΔK1, ΔK3, ΔK1/K3, Y74F, and Y88/89F cassettes were cloned into the pTRE3G tetracycline inducible retroviral expression construct using the In Fusion Gene Cloning kit (Clontech). Alt Brk was generated by PCR using human Alt-Brk in PCDNA3 vector (38) as a template, followed by cloning into the T7pGEMEXhuman Flag-tagged plasmid and pTRE3G using the In-fusion cloning kit. The amino acid sequence of Alt-Brk is shown below:

(SEQ ID NO: 12)
MVSRDQAHLGPKYVGLWDFKSRTDEELSFRAGDVFHVARKEEQWWWAT
LLDEAGGAVAQGYVPHNYLAERETVESEPAGHAGCAALQDLAACRGPA
APERGGVLPQPARACELPQGPEPVPRPAAGRALPEARA.

Mimetics and mutants of this sequence can be generated by truncation of 3, 5, 10, 15 20, 25, 50 or more amino acids. Variants in which individual amino acids can be substituted by other amino acids which are closely related can also be generated. For example, individual amino acid may be substituted as follows: any hydrophobic aliphatic amino acid may be substituted in place of any other hydrophobic aliphatic amino acid; any hydrophobic aromatic amino acid may be substituted in place of any other hydrophobic aromatic amino acid; any neutral amino acid with a polar side chain may be substituted in place of any other neutral amino acid with a polar side chain; an acidic amino acid may be substituted in place of an acidic amino acid; and a basic amino acid may be substituted in place of a basic amino acid. Variants and mutants having preferred properties and activities can also be generated by substitution of certain amino acids with amino acids that are not closely related, for example replacing a charged amino acid with a neutral amino acid. Fusion proteins of non contiguous amino acids could also generated. Specific alterations can be made using information available from previously solved 3D structures of a variety of Src family tyrosine kinases. Each of the mimetics should be effective to interfere with pY phosphorylation.

### Recombinant Cyclin D1-Cdk4.

Recombinant His-cyclin D1-cdk4 was harvested from co-infected HighS cells and purified as described previously (James et al., supra). Recombinant GST-Rb (86 Kdversion) was purified and used in in vitro kinase assays.

In Vitro Phosphorylation of the p27-Cyclin D1-Cdk4 Ternary Complex.

Recombinant His-p27 and mutants were incubated for one hour at room temperature with Cyclin D1-Cdk4 in 25 mM Hepes, pH 7.4. This ternary complex was immunoprecipitated with anti-Cdk4 antibodies (Santa Cruz, DCS 35) and Protein G Dynabeads (Invitrogen, 10004D). The complex was then subjected to SFK phosphorylation and/or used in in vitro Rb kinase assays.

### Cell Lines.

MCF10A, MCF7, MDA MB 231, MDA MB 468, T47D, PC3, Mv1Lu and HEK 293 were purchased from ATCC and maintained according to vendor's instructions. Insulin levels were adjusted to 0 (−), 10 (+) or 50 (+++) μg/ml and cells were grown for 2 weeks before being assayed as described. To arrest by contact, cells were grown to confluence and maintained for 6 days, replenishing the media every other day. Immunoprecipitation, immunofluorescence, PI staining were performed as described in materials and methods section. FACS analysis was performed as described (Nguyen K. D. et al. (2010) J. Pediatr. Gastroeneterol. Nutr. 5, 556-62). Cells were counted using the automated cell counter (BioRad TC-20). Viability was measured by Trypan Blue staining and counted using the cell counter.

### Immunoprecipitation.

Cells were either lysed with Triton lysis buffer (25 mM HEPES pH 7.4, 100 mM NaCl, 1 mM EDTA, 10% Glycerol, 1% Triton X-100) or Tween lysis buffer (50 mM HEPES pH 7.4, 150 mM NaCl, 1 mM EDTA, 2.5 mM EGTA, 10% Glycerol, 0.1% Tween-20). The lysis buffers were supplemented with 1 mM PMSF, 10 mM DTT, 1 mM NaV, 10 ng/ml Leupeptin and 1 ng/ml Aprotinin. Lysates (1 mg) were pre-cleared by incubation with Dynabeads A or G (Life Technologies) for 1 h at 4° C. Immunoprecipitations proceeded as described (James et al., supra).

### Immunofluorescence.

Cell lines were split on day 0 into sub-confluent conditions and fixed on day 2 in microwell plates using 4% paraformaldehyde in 1×PBS, pH 7.4, for 15 min at room temperature. They were permeabilized with 0.1% Triton X-100 and blocked with 5% BSA for 1 h at room temperature. They were incubated with the first round of primary antibodies in PBS for 1 hour at room temperature. The cells were washed with PBS and incubated with appropriate secondary antibodies (1:500), diluted in 3% BSA/PBS, for 1 hour at room temperature. They were then washed with PBS and incubated with 0.02% Triton X100/3% BSA for 30 min at room temperature to prepare them for a second round of incubation with antibodies. Cells were then washed with PBS and incubated with Hoechst stain (1 mg/ml) 1:5000 in PBS for 15 min at room temperature. They were rinsed with water and mounted on a slide with 90% gylcerol. Samples were incubated at 4° C. before they were analyzed by confocal microscopy.

### Inhibitor Treatment.

Cells were seeded on six well plates in duplicate, 5.0×10⁴ per well. 24 hours post seeding, one well for each plate was treated with trypsin and counted using the Biorad Automated cell counter. 48 hours post seeding, another well was treated with trypsin and counted and the rest of the wells were treated with Palbociclib (SelleckChem) at 50 nM, 100 nM, 200 nM and 400 nM. DMSO was used as a negative control. Cells were counted again 24 and 48 h post treatment. The IC₅₀ values were determined by normalizing the number of viable cells treated with different concentrations of Palbociclib to the number of viable cells treated with DMSO for each cell line 48 hours post treatment. The number of viable cells treated with DMSO was considered 100%. The log of the viability values was obtained and the data was fitted to a nonlinear regression curve, which was used to generate the IC₅₀ values using Graphpad Prism software.

### Brk Knockdown.

Lentiviral siRNA particles were purchased from Sigma Aldrich:NM_005975.2-1064sc1 and NM_004383.x-2117s1c1. MCF7 cells were plated on day 0, on day 1, the media was aspirated and the cells were infected with the siRNA lentiviral particles. Hexadimethrine bromide was used according to manufacturer's instructions to enhance the infection efficiency. Cells were incubated overnight, media was replenished on day 2 and the cells were incubated for 72 hrs, fixed with 4% Paraformaldehyde and immunofluorescence was performed as described.

### Expression In Vivo.

Generation of the WT-Brk, KM-Brk, and YF-Brk has been described (Palka-Hamblin H. L. et al., (010) J. Cell Sc. 123: 236-45). Amphotropic retroviruses were generated by transfection using Lipofectamine 2000 (Life Technologies 11668-019) of HEK 293 cells with pAmpho envelope and pBabe or pTRE3G tetracycline inducible constructs. Following viral infection of MCF7 cells, stable integrants were isolated by puromycin selection. Colonies were pooled to generate stable, puromycin resistant clones. Stable expression was verified by immunoblot and immunofluorescence analysis. Tetracycline inducible expression was achieved by the addition of TetExpress (Clontech) to the media.

### Quantitative RT-PCR.

RNA extraction was performed using TRIzol reagent (Life Technologies) as directed by the manufacturer's instructions. 500 μg of RNA was subjected to reverse transcription using the Verso cDNA kit (Thermo Scientific). 250 ng RNA was mixed with cDNA primers and ABsolute Blue qPCR SYBR Green (Thermo Scientific) to perform qPCR.

Following Primers were Used to Perform q-PCR:

| GENE FORWARD PRIMER | | REVERSE PRIMER |
|---|---|---|
| Actin | 5′-AAAATCTGGCACCACACCTTCTAC-3′(13) | 5′-TAGCACAGCCTGGATAGCAACG-3′(14) |
| Brk | 5′-CCAAGTATGTGGGCCTCTGG-3′(15) | 5′-AAAGAACCACGGTTCCGACT-3′(16) |
| Alt Brk | 5′-GACGGTGGAGTCGGAACCTG-3′(17) | 5′-TAGTTCACAAGCTCGGGCAG-3′(18) |

(Numbers in parentheses are SEQ ID NOS).

### Analysis of Human Patient Material.

Material discarded from lumpectomy or mastectomy from ER/PR+, Her2− patients obtained with Informed Consent from patients at University Hospital, Brooklyn, was grown in explant culture for 48 h. in DMSO, a high non-physiological Palbociclib, or a physiological concentration of Palbociclib (purple). Six 1 mm³ sections of patient material were placed in wells of a 6 well dish on a dental sponge saturated in warm complete DMEM media+FBS. Samples were allowed to recover for 48 h. in the incubator. After 48 h. was DMSO, 100 nM Palbociclib, or 500 nM Palbociclib was added for 48 more h. The explant sample was removed with forceps and material was fixed in 10% formalin, paraffin embedded and stained by IHC for Ki67, as a marker of proliferation. Palbociclib response was measured as a decrease in Ki67 in the presence of 100 nM Palbociclib. Each data point is the average of 4 samples (2 independent explant samples, and 2 independent immunohistochemistry stainings of each explant (runs A and B). Each sample was read blindly by two pathologists.

Biopsy or resection material removed from the same patients at the time of lumpectomy or mastectomy was sent to DMC Pathology Department for fixing in 10% formalin and paraffin embedding. Material was then stained in the dual pY/p27 IHC assay as described.

### Scale:

0=no pY staining
1=1-29% pY+ cells, with 0% strong staining
2=1-29% pY+ cells, with only 5-20% strong staining
3=30-100% pY+ cells, with >20% strong staining

### Cell Block Preparation.

1×10¹² cells were grown in tissue culture, spun down and fixed in 10% formalin, embedded in paraffin, and then analyzed in the dual pY/p27 IHC assay as described. MCF7 cells were treated with 400 nM Palbociclib for 48 h. before cell block preparation. Five independent experiments were read blindly by two pathologists.

### Scale:

0=no pY staining
1=weak pY staining
2=moderate pY staining
3=strong pY staining
Dual Immunohistochemistry Assay with p27/pY88 Antibodies.

### Reagents:

STAINING KIT: Enzo Lifesciences (ADI-950-100-0001); Antigen Retrieval Solution: Dako (S1699); PAP Pen: Fisher Scientific (XT001-PP); Protein Block: Dako (X0909); Antibody Diluent: Dako (S3022); P27 Antibody: BD Biosciences (610242); Mounting Solution: Fisher Scientific (SP15-100)

### Staining Protocol

On day 1, slides are labeled with a pencil and baked in an oven at 65° C. for 30 minutes and immediately transferred from the oven to a coplin jar containing Xylene. Slides are rinsed in Xylene 4× for 3 minutes each at room temperature followed by a graded alcohol wash at 100%-95%-75% ethanol 3 times, 3 min each at R.T. Slides were then hydrated by incubation in H₂O once for 3 min. After drying, a hydrophobic barrier was created around the tissue using a PAP pen. Endogenous peroxide was blocked by incubating the slides with Peroxide block for 30 min at room temp. The slides were washed with TBS-0.1% Tween 20 3 times for 3 minutes each followed by incubation in 1×PBS for 3 min. After an incubation with a with protein block for 1 h at R.T, P27 pY88 antibody diluted 1:200 in the DAKO antibody diluent was added to the slides for an overnight incubation at 4° C.

On day 2, a 1× solution of antigen retrieval was prepared from 10× stock and it was equilibrated at 100° C. in the water bath. After the temperature of antigen retrieval solution reached 100° C., the slides were washed with TBS-0.1% Tween 20 3 times, 3 min each at R.T. Antigen retrieval was performed at 100° C. for 30 min. After 30 min, the coplin jar was allowed to cool down at R.T. for another 20 min. The slides were then incubated in 1×PBS for 3 min. P27kip1 antibody (1:1000) dilution was prepared in the DAKO antibody diluent. Slides were incubated with p27kip1 antibody overnight at 4° C.

On Day 3, the slides were incubated in TBS-0.1% Tween 20 3 times, 3 min each. Polyview IHC Mouse HRP and Polyview IHC Rabbit AP were mixed in equal volumes in an Eppendorf and the mixture added to the slides and incubated for 20 min at R.T. The slides were then incubated in TBS-0.1% Tween 20, 3 times, 3 min each. During this incubation period, 1 ml of Mouse HRP chromogen buffer was mixed with 20 ul (or one drop) DAB chromogen. They were mixed by inverting and protected from light. Slides were incubated with the activated DAB substrate for 5 min at R.T. They were washed in TBS-0.1% Tween 20 3 times, 3 min each. 1 ml Rabbit AP chromogen buffer was mixed with 20 ul (or one drop) of AP chromogen by inverting the tube and protecting it from light. Slides were then incubated with the activated AP substrate for 15 min at R.T and washed in TBS-0.1% Tween 20 3 times, 3 min each followed by a tap water wash for 5 min at R.T. Slides where then rinsed with 75%-95%-100% ethanol 3 times, 3 min each at R.T. and mounted using Permount solution.

### Statistics.

The statistical analysis was performed using the Student's t test, Welch's t test, 2 tailed type 3 test, due to unequal sample sizes with unequal variances.

The following examples are provided to illustrate certain embodiments of the invention. They are not intended to limit the invention in any way.

### Example I

### PY as a Marker for Determining Cancer Cell Sensitivity to CDK4 Inhibitors

In the present example, pY is used as a marker in human patient material to determine whether a particular tumor has the appropriate range of cdk4 activity suitable for inhibition by cdk4 inhibitors. We have developed a phosphospecific antibody for pY p27 and shown that it recognized pY in formalin fixed, paraffin embedded archival human breast cancer material (ER/PR+/Her2−). With 100% penetrance, the antibody did not stain benign tissue obtained from human mammary reduction surgery. 75% of the ER/PR+/Her-2 breast cancer samples analyzed stained positive for pY albeit with different intensities.

p27 contains three putative SH3 recruitment sequences that contain the common PxxP core motif, designated K1, K2 and K3 (FIG. 1A). K1 contains a basic residue after the PxxP, thus qualifying it as a canonical type 2K SH3 target site (Cesareni G. et al., (2002) FEBS Lett. 513: 38-44). K2 is only present in the human orthologue of p27 and thus is unlikely to mediate conserved functions in cell cycle control. K3 is at the C-terminus of p27, in a region that has shown to be dispensable for cdk interaction. Based on the reported interactions of p27 with non-receptor bound tyrosine kinases (SFKs), such as Src, Yes, and Lyn, we asked whether other members of the family might also interact with p27, and which recruitment sequences (K1, K2, and/or K3) are used. We tested 11 members of the SFK family as well as Abl, which has been reported to phosphorylate p27 in vitro and in vivo, for binding to either full-length p27, or GST-tagged K1, K2 or K3 peptides, using a phage-ELISA procedure (Asbach B. et al. (2012) PLoS One 6: e38540.) (FIG. 1B, 1C).

While the SH3 domains of most SFKs could interact with full-length p27, we found that the SH3 domain of Brk interacted strongly with full-length p27 (Kd=250 nM), and associated better than either Src or Abl, two SFKs known to interact with p27 (FIG. 1B). This Kd value is reflective of the interaction between p27 and the phages, which contain many identical reiterated SH3 domains, which would enhance binding. We expressed the individual SH3 recruitment sites within p27 (K1, K2 and K3) as GST-fusion peptides and tested them against the SH3 domain library (FIG. 1C). The GST domain expressed in the absence of any p27 sequence was used as a negative binding control (GST). Most SFK SH3 domains were not able to interact significantly with the individual PxxP-containing peptides (data not shown). In this assay Brk interacted strongly with the K3 region, and weakly with the K1 region (FIG. 1C). The related kinase, Frk, was the second best binder to full length p27 (FIG. 1B), but when tested against the individual PxxP domains, significant binding to the GST negative control was detected (FIG. 1C), so we could not conclude whether Frk's SH3 domain bound to the PxxP domain peptides. The SH3 domains of Abl and Yes interacted with the K3 domain of p27, although this interaction was reduced when compared to that of Brk (FIG. 1C). No SH3 domains interacted with the K2 site (FIG. 1C). The sequence of Brk with the SH3 domain highlighted is provided in FIG. 1D. We have performed in silico modeling analysis with the Brk and Src SH3 domains, using the 3D structures, derived from X-ray crystallographic studies, and taken from the PDB+jFATCAT rigid databases. We determined that differences in the loops connecting the beta sheets existed between the Brk SH3 domain, which binds p27, and the Src SH3 domain, which doesn't bind p27. When we swapped the Src SH3 loops into the Brk Sh3 structure we created a variant that didn't bind p27, demonstrating that at least in part, some specificity was derived from the loops. We identified residues in the Brk SH3 domain loops that mediated this binding and substituted them for residues to increase affinity between the Brk SH3 variant and the p27 K1 domain. When variants of p27 were generated with altered K1 or K3 sites, the results showed that the K1 site was required for pY88 phosphorylation both in vitro and in vivo. While the K3 site might bind the Brk SH3 domain better as a monomer peptide, in the context of the full length p27, binding and phosphorylation is mediated through the K1 site.

Cyclin D-cdk4 (DK4) has been a highly sought after therapeutic target because it drives cancer proliferation in a majority of human tumors, including ER/PR+, Her2− breast cancer. We have explored the clinical utility of a recently discovered mechanism of cell cycle control exerted on DK4 by p27Kip1 and its activator, the Breast tumor Related Kinase (Brk), in predicting responsiveness to therapy and as a new target for treatment. Although known as a DK4 assembly factor and cdk2 inhibitor, p27 also acts as a DK4 ON/OFF “switch.” Tyrosine (Y) phosphorylation of p27 (pY) by Brk controls both ATP binding and CAK phosphorylation of cdk4's T loop, which are essential for DK4 activation. This function is restricted to cdk4: p27's association with cdk2, whether Y phosphorylated or not, appears to be inhibitory. However, in vivo Y phosphorylated p27 is a target for cdk2-dependent ubiquitin-mediated degradation, reducing p27's association with cdk2, indirectly activating this complex. This leads to the following model: blocking p27 pY would inactivate cdk4 directly and cdk2 indirectly, and thus represents a novel way to block cancer cell proliferation. pY also serves as a predictive biomarker of cdk4 inhibitor activity, tumor response to therapy and chemo-resistance.

To block pY in breast cancer cell lines, we used a small peptide ALT, which contains a portion of Brk's SH3 domain. ALT binds to p27, blocks Brk's association and ability to phosphorylate p27, inhibiting cdk4 and increasing p27's ability to inhibit cdk2. We generated Tetracycline inducible cell lines that expressed ALT and/or engineered a lipid-based nanoparticle delivery vehicle (NP-ALT), permitting us to test ALT as a first generation therapeutic. ALT was also used with Palbociclib to determine if combination therapy reduced drug resistance. pY inhibition comes with a built in biomarker for efficacy and the identification of responsive cell lines and/or future patients. We developed a dual IHC assay for p27 and pY, which we used to analyze both a breast cancer cell line panel previously stratified for Palbociclib sensitivity as well as paraffin-embedded, archival human tumor samples.

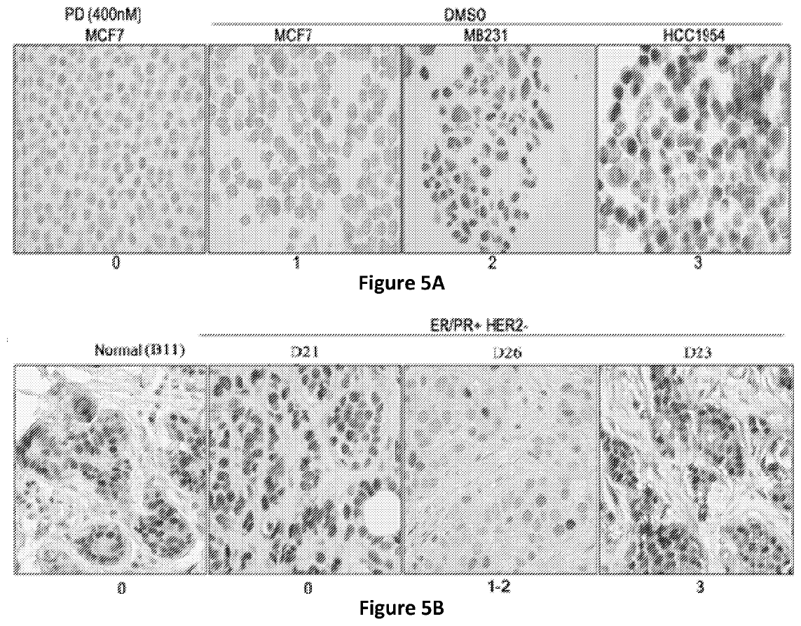

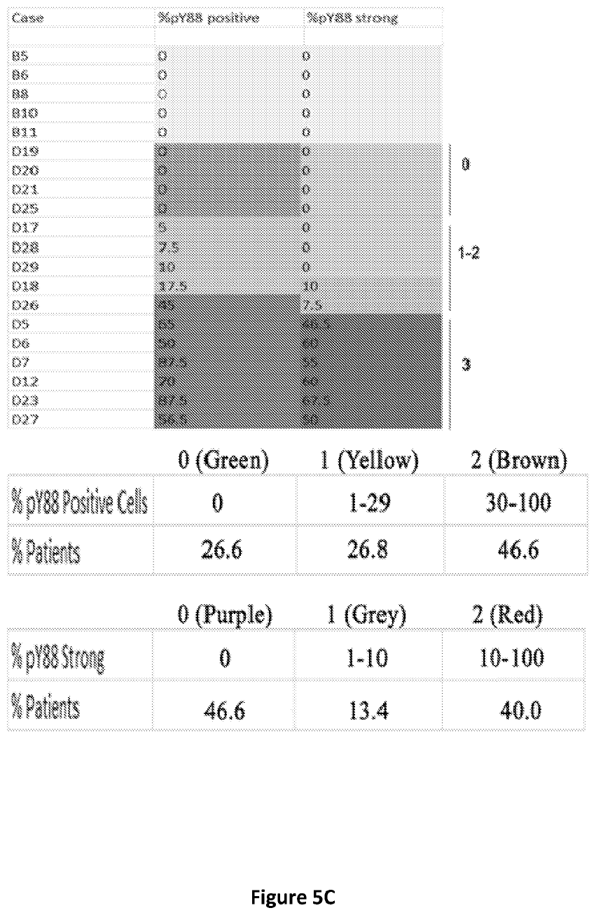

Our results show that ALT blocked pY, cdk4 and cdk2 activity, and proliferation in ER/PR+, Her2− breast cancer cell lines. Palbociclib-mediated arrest in several lines is not very durable and cells quickly become resistant to therapy, and we demonstrated that this is due to the ability of cdk2 to compensate for loss of cdk4 activity. Since ALT inactivates both cdk4 and cdk2, Alt-mediated arrest is more drug resistant (arrest for >10 days). As a dual therapy, ALT treatment synergized with Palbociclib to arrest cells for >30 days, and increased senescence, preventing recovery post drug removal. pY levels correlated with cdk4 activity: increasing or decreasing Brk expression increased or decreased pY and cdk4 activity respectively, which correlated with increased or decreased Palbociclib sensitivity. We found that MCF7 cells, which respond well to Palbociclib (IC₅₀=200 nM) had lower pY (less cdk4 activity requiring less drug), while Rb+ cells, like HCC1954 which did not respond well to Palbociclib administered in the therapeutic window, had very high pY, indicating higher cdk4 activity requiring elevated concentrations of drug (IC₅₀=1000 nM). See FIG. 5. Cells like MDA MB231, which had an intermediate level of pY, had an intermediate requirement for Palbociclib (IC₅₀=400 nM). Analysis of human cancers obtained from archival sources, demonstrated that pY is never detected in quiescent benign mammary tissue, but is detected in about half of the advanced ER/PR+/Her2− tumors analyzed, albeit with different staining intensities.

We conclude that blocking p27 pY provides a powerful approach for inhibiting Cd4k inhibitor sensitivity and cancer cell proliferation because it inhibits both cdk2 and cdk4, induces senescence and prevents drug resistance. Our data suggest that while the level of cdk4 activity, as measured by pY, will determine initial responsiveness, to inhibit drug resistance, cdk2 activity should also be inhibited. It is clear from the above that pY88 levels can be used to predict cd4k inhibitor sensitivity, where a level of 0 indicates no detectable sensitivity to cdk4 inhibition, a level of 1 indicates low or no detectable sensitivity, and a 2 or 3 indicates detectable sensitivity to cdk4 inhibition.

### The Brk-p27-DK4 Axis is a Predictive Biomarker of Cdk4 and Cdk2 Activity, Tumor Response and Resistance.

There is an urgent need to identify patients that will respond to Palbociclib and other cdk4 selective inhibitors. While PFS was significantly improved when patients were treated with Palbociclib and Letrozole, overall survival (OS) was not statistically different. In this study, complete non-responders were not eliminated from the OS data. We hypothesize that if non-responders could have been identified and were removed from the trial, clinical outcomes might have been better. Inasmuch as Palbociclib costs >$100K per year, identification of potentially responsive patients is a desirable goal.

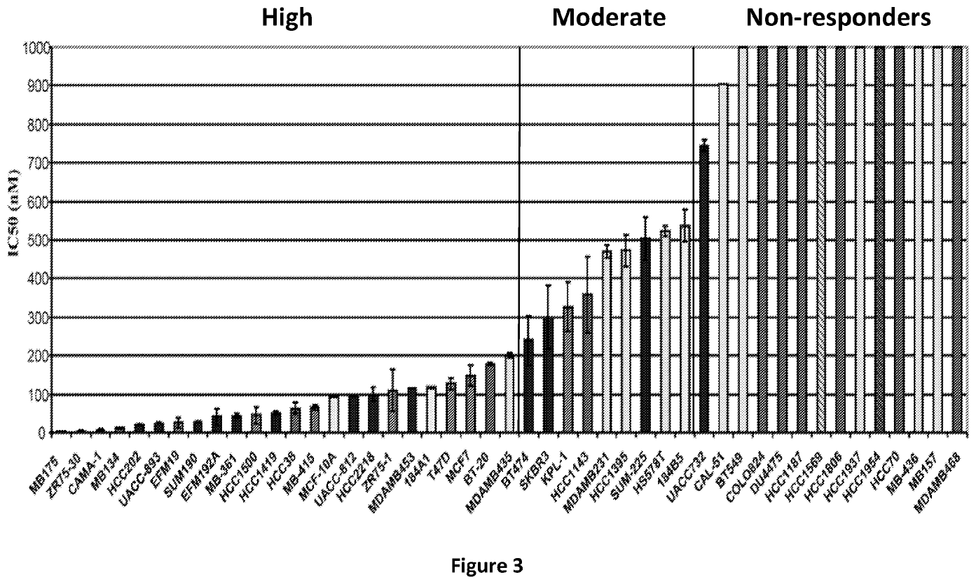

In vitro large-panel analyses of molecularly characterized breast cancer cell lines provide insight into which subgroups will be more likely to benefit from cdk4 blocking therapy. Breast cancer cell lines have been stratified for Palbociclib sensitivity (FIG. 3). IC₅₀ values of response are shown, and lines can be subdivided into high, moderate or non-responders. In this study, several resistant lines that did not contain RB were identified, and thus inhibiting cdk4 had no effect. However, there were also several Rb+ resistant lines, such HCC1186, HCC1954, and Cal51. In these lines, Palbociclib did not inhibit cdk4-dependent RB phosphorylation, suggesting that either CDK4 was mutated in such a way as to prevent Palbociclib association, or the level of cdk4 activity was too high (≥3) to allow inhibition by the drug in the therapeutic window, or some other kinase, such as cdk2, is compensating for cdk4 loss, permitting Rb phosphorylation. Notably, while cell lines can be used to predict efficacy of anti-cancer agents, data obtained in cell lines can significantly differ from that observed in tumors in situ or in tumor ex vivo.

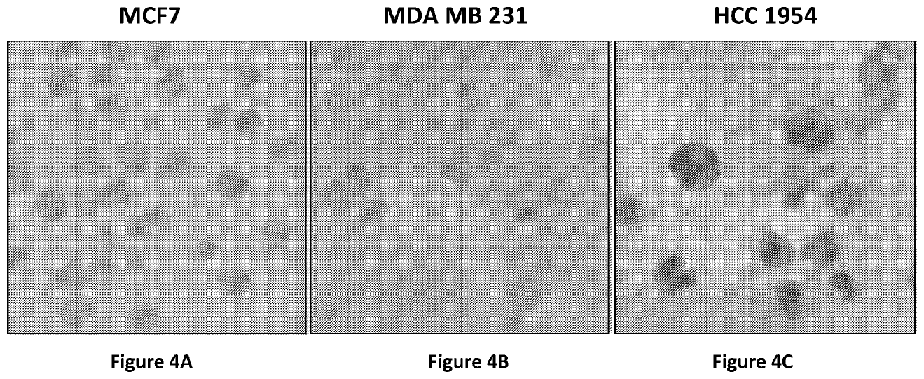

We hypothesized that pY levels might stratify with Palbociclib sensitivity. We tested one each of the non-responder (HCC1954), moderate (MDA MB 231), and high-responders (MCF7) cell lines by dual IHC for p27 (brown) and pY88 (pink) expression (combining the DAB detection system with APAAP) (FIG. 4). While we detected pY88 in all three lines, we found that the high responder (MCF7, IC₅₀=200 nm) had lower pY staining (with +1 staining intensity), while the moderate responder (MDA MB 231, IC₅₀=400 nm) had mid-levels of pY (with +1-2 staining intensity), and the non-responder (HCC1954, IC₅₀=1000 nm) had very high levels of pY88 (with +2-3 staining intensity). We might have expected to see complete lack of pY88 in the non-responder line, indicating that there wasn't any cdk4 present as a target for the drug. However we detected the most intense pY88 staining in this line. This data suggest that the level of cdk4 may dictate arrest and a “speed limit” type model might explain these results: too little cdk4 as indicated by no pY staining (level 0) identifies complete non-responders, but too much cdk4 (level ≥3), as indicated by very high pY staining, can also identify non-responders because the concentration of Palbociclib needed to show a response in vivo, and inhibit this amount of cdk4, is toxic (>1000 nm). In this instance, a different cdk4 inhibitor (described herein below) may function better than Palbociclib to inhibit cancer cell proliferation. Notably, when MCF7 cells were treated with 400 nM Palbociclib, pY staining decreased (0% of cells stained) and resulted in a 0 intensity.

Breast cancer cells become resistant to Palbociclib treatment with varying kinetics, and our data suggest that long term Palbociclib response vs. drug resistance may be due to the cdk2 activation. Palbociclib may be able to initially and transiently inhibit cdk4 (cell cycle arrest), but with time, cdk2 is able to compensate for this loss and overcome cytostasis (resistance). Increasing or decreasing pY will “toggle” Palbociclib sensitivity up or down. As we have shown in MCF7 cells, inhibiting Brk reduces p27 pY and reduces cdk4 activity. Conversely, increasing Brk expression increases p27 pY and increases cdk4 activity.

We have screened 15 cases of formalin-fixed, paraffin-embedded breast tumors, obtained by needle biopsy (FIG. 5). These tumors were Grade II and III, ER/PR+, Her2−, a subgroup with poor outcome. This subgroup accounts for approximately 40% of breast cancer patients, and they are not candidates for Her2− targeting therapy. This subgroup is currently the focus of the Palbociclib Paloma trial(s). All samples were grade 2-3 and were Ki67+ ranging from 10-50%. No other differences could be detected among this sample set by pathology. However, by staining in the p27/pY88 assay, we were able to further stratify these otherwise indistinguishable ER/PR+, Her2− samples, based on their pY status. We also screened 5 benign mammary biopsies. We performed dual IHC analysis with p27 (brown) and pY88 (pink) antibodies and results were blindly analyzed by two independent pathologists (FIG. 5B, C). Slides were scored for percentage of cells staining positive, and intensity of the pY88 stain (0, 1, 2, 3). We found 100% of the non-neoplastic, normal samples were positive for p27 and negative for pY88, consistent with the model that quiescent tissue has inactive cdk4 (FIG. 5B, 5C, creme box). These samples were Ki67− (quiescent) and lack pY staining suggesting that cdk4 was inactive and the cells were not in cycle. This also confirmed that the pY88 antibody is specific for Y phosphorylation, and does not recognize non-phosphorylated p27. As shown in Table 1, 100% of the ER/PR+, Her2− tumors analyzed were positive for p27 staining. Three stratifications of pY88 were detected in this tumor subgroup: 47% had high pY88 staining, 25.5% had low and focal pY88 staining and 25.5% had no pY88 staining.

TABLE 1
Her2-patients

| | |
|---|---|
| pY subgroup | |
| None | 25.5% |
| Moderate | 25.5% |
| High | 47% |
| None: | 0% of cells + pY |
| Moderate: | 1-29% of cells + pY |
| High: | 30-100% of cells + pY |

While we do not have corresponding Palbociclib sensitivity data for these patients, the results demonstrate that this subgroup can be further divided based on pY levels, which would translate into cdk4 activity levels indicative of differential sensitivity to Palbociclib. The big difference detected between the cell lines and the data here is that 25.5% of patients had no pY88 staining (level 0). This is not unexpected, as cell lines are proliferative and transformed. Our data indicate that the 0 pY88 staining group will not respond to the Palbociclib.

The second difference was that while the cell lines were clonal and every cell stained the same way, due to inherent tumor heterogeneity in the same tumor, in the tumor block, some cells stained and some did not. We calculated the percent of cells that stained + (above), but we also determined pY88 intensity on a scale of 0-3 (FIG. 5A), where 0 indicates Y88 negativity, and was more similar to the scale used in the cell blocks. 26.7% of the samples were negative for pY staining (FIG. 5B, green, 0) and resembled the normal material (creme). This indicates that this group does not have active cdk4, and would not respond to PD treatment. The rest of the samples did have cells that stained with pY: 26.7% of samples had between 1-29% of the cells staining for pY (FIG. 5B, yellow, levels 1-2), and 46.6% had between 30-100% staining (FIG. 5B, brown, level 3). In group 3, where >30% of cells stained for pY, ˜of 0% of the stained cells stained strongly for pY (% pY88 strong, red). One patient (D26), however had many cells that were Y88+, but only 7.5% were Y88 strong. In group 1 (FIG. 5B, yellow), where fewer cells were positive, most did not stain strongly for pY88 (FIG. 5B, purple), but two (D18 and D26) had a few <10% strongly staining cells (FIG. 5B, grey). Samples that fell into the 0 and 3 groups were easily distinguished: group 0 had no Y88 staining and no strong Y88 staining, while group 3 had >30% cells staining with Y88 and >50% of those cells stained strongly. The 1-2 group was more mixed and could be divided itself into 2 groups: those that had <10% cells staining weakly positive (no strong Y88 staining) and those that stained between 20-50% positive, and had <20% staining strongly. The data indicate that the 0 group would not respond to PD treatment, as these samples lacked pY and thus lacked cdk4 activity. Group 3 should respond to PD because the “druggable” target is present and indicative of cdk4 activity. The level of pY in group 3 can be correlated with a level of cdk4 activity that may require higher concentrations of Palbociclib. Samples falling into group 1-2 would require lower Palbociclib concentrations.

FIG. 7 shows the results from explant cultures of material discarded from lumpectomy or mastectomy from ER/PR+, Her2− patients treated with no (DMSO; green), high non-physiological Palbociclib (500 nM, red), or a physiological concentration of Palbociclib (100 nM, purple). Fixed, paraffin embedded blocks were stained for Ki67, as a marker of proliferation. The high concentration of drug (purple) was an internal control that proliferation could be inhibited. Each patient had an inherent different proliferation rate as measured by different Ki67 levels in the untreated sample (green). Palbociclib response was measured as a decrease in Ki67 in the presence of the physiological concentration of drug (red). Patients 1 and 3 responded. Each data point is the average of 4 samples (2 independent explant samples, and 2 independent immunohistochemistry stainings). Each sample was read blindly by two pathologists.

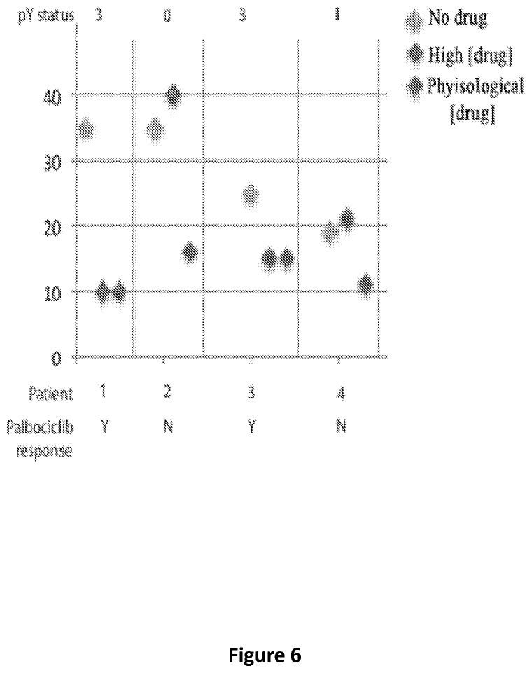

In a separate experiment, material removed from the same patients at the time of from lumpectomy or mastectomy was sent to DMC pathology Department for fixing and paraffin embedding. Material was then stained in the dual pY/p27 IHC assay. Samples were scored as FIG. 6.

0=no pY staining

1=1-29% pY+ cells, with 0% strong staining

2=1-29% pY+ cells, with only 5-20% strong staining

3=30-100% pY+ cells, with >20% strong staining

From this experiment (n=4 patients), pY status of 3=response, while pY status of 0 or 1=no response. In the sample staining with an intensity of 1, it is possible that the tumor block lacked a sufficient number of cells harboring the cdk4 target or that the tumor is less dependent on cdk4 activity.

We have developed a very convenient, reproducible assay, and analysis can be rapidly applied to these other breast cancer subgroups and other cancer types. Our assay can be applied to resection material, which typically has both malignant and benign regions as defined by architectural characterization. We will determine whether breast cancer patient-derived benign regions (particularly at the margins of resection) are as “benign” as material obtained from non-breast cancer patients. As pY88 clearly is absent from benign tissue, its detection in breast cancer patient-derived benign regions will demonstrate that margins are not as “clean” as architectural characterization might suggest.

pY can also be used as a biomarker to predict response in patient material obtained with IRB approval from biopsy and lumpectomy procedures. A dose response curve with three concentrations of Palbociclib is used to determine IC₅₀ values. IC₅₀<200 nM will be defined as high response. IC₅₀ between 201 nM-500 nM as moderate, and, IC₅₀ greater than 500 nM as non-responsive.

### Example II

### Test and Treat Method for Ameliorating Spread and Symptoms Associated with Cancer, Particularly Breast Cancer

In another aspect of the invention, a test and treat method is disclosed. First, a sample is taken from the tumor and Y88 phosphorylation levels assessed in order to determine cdk4 activity levels. As described at length in previous examples, patients having no detectable levels of Y88 phosphorylation relative to levels observed in normal tissues, are not likely to benefit from cdk4 inhibitor therapy, while patients having levels of 1, 2 or 3 of pY88 phosphorylation relative to levels observed in normal tissues, should benefit from cdk4 inhibitor therapy at differing concentrations. In order to treat an individual having cancer to alleviate a sign or symptom of the disease, suitable agents targeting cdk2 and cdk4 disclosed in the Table 2 and described in the Examples above, can be administered in patients most likely to benefit, alone or in combination in order to reduce tumor burden in the patient. Such agents should be administered at the effective dose. The total treatment dose or doses (when two or more targets are to be modulated) can be administered to a subject as a single dose or can be administered using a fractionated treatment protocol, in which multiple/separate doses are administered over a more prolonged period of time, for example, over the period of a day to allow administration of a daily dosage or over a longer period of time to administer a dose over a desired period of time.

One skilled in the art would know that the amount of cdk inhibitor required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having cancer, particularly breast cancer.

The effective dose of cdk inhibitor will depend on the mode of administration, and the weight of the individual being treated. In an individual suffering from cancer, in particular a more severe form of the disease, administration of cdk inhibitors can be particularly useful when administered in combination, for example, with a conventional agent for treating such a disease. The skilled artisan would administer the therapeutic agent(s), alone or in combination and would monitor the effectiveness of such treatment using routine methods such as radiologic, immunologic assays, or, where indicated, histopathologic methods. In preferred embodiments, Y88 phosphorylation levels can be used to monitor effectiveness of treatment over time.

In a preferred embodiment of this invention, a method is provided for the synergistic treatment of cancer using the pharmaceutical agents disclosed in the present example in combinatorial approaches. As described above, Alt-Brk (or another agent which interferes with Y88 phosphorylation) in combination with Palbociclib effectively synergize to arrest breast cancer cell proliferation. Advantageously, the synergistic method of this invention reduces the progression of cancer, or reduces symptoms associated with cancer in a mammalian host. The information provided herein guides the clinician in new treatment modalities for the management of breast cancer.

Methods for the safe and effective administration of most of these agents are known to those skilled in the art. In addition, their administration is described in the standard literature. For example, the administration of many of the anti-cancer agents is described in the “Physicians' Desk Reference” (PDR), e.g., 1996 edition (Medical Economics Company, Montvale, N.J. 07645-1742, USA); the disclosure of which is incorporated herein by reference thereto.

The present invention also encompasses a pharmaceutical composition useful in the treatment of cancer, comprising the administration of a therapeutically effective amount of the combinations of this invention, with or without pharmaceutically acceptable carriers or diluents. The synergistic pharmaceutical compositions of this invention comprise two or more of the agents described in the previous examples, and/or listed in the table below and a pharmaceutically acceptable carrier. The compositions of the present invention may further comprise one or more pharmaceutically acceptable additional ingredient(s) such as alum, stabilizers, antimicrobial agents, buffers, coloring agents, flavoring agents, adjuvants, and the like. The anti-cancer compositions of the present invention may be administered orally or parenterally including the intravenous, intramuscular, intraperitoneal, subcutaneous, rectal and topical routes of administration.

Certain cancers can be treated effectively with a plurality of the compounds listed above. Such triple and quadruple combinations can provide greater efficacy. When used in such triple and quadruple combinations the dosages can be determined according to known protocols.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

Also, in general, the compounds described herein do not have to be administered in the same pharmaceutical composition, and may, because of different physical and chemical characteristics, have to be administered by different routes. For example, first compound may be administered orally to generate and maintain good blood levels thereof, while a second compound may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

As described previously, the present inventor has identified a new marker for predicting long term survival and response to cancer therapy in breast cancer patients. CDKtargets with known drugs available are shown in Table 2. These drugs can be combined to synergistically treat cancer or to simultaneously reduce symptoms or progression of cancer, particularly breast cancer.

TABLE 2

| Inhibitor (company) | Main targets (other targets) |
|---|---|
| AG-024322 (Pfizer)^{‡} | CDK1, CDK2 and CDK4 (other CDKs) |
| AT7519 (Astex) ^{‡} | CDK2, CDK4, CDK5 and CDK9 (CDK1, |
| | CDK4, CDK6 and GSK3β) |
| AZD5438 (AstraZeneca) * | NA |
| Flavopiridol, also known as | CDK1, CDK2, CDK4, CDK6, CDK7 and CDK9 |
| alvocidib (Sanofi-Aventis) ^{‡} | (GSK3β) |
| Indisulam, also known as E7070 | NA |
| (Eisai) ^{‡§} | |
| P1446A-05 (Nicholas Piramal)* | CDK4 (NA) |
| P276-00 (Nicholas Piramal) ^{‡} | CDK1, CDK4 and CDK9 (CDK2, CDK6 and CDK7) |
| PD-0332991Palbociclib (Pfizer)* | CDK4 and CDK6 (NA) |
| R-roscovitine, also known as | CDK1, CDK2, CDK5, CDK7 and CDK9 (CK1, |
| CYC202 and seliciclib (Cyclacel)* | GSK3α-β, DYRK1A, ERK1, ERK2 and PDXK) |
| R547, also known as Ro-4584820 | CDK1, CDK2, CDK4 and CDK7 (NA) |
| (Hoffmann-La Roche) ^{‡} | |
| SCH 727965 (Schering-Plough) ^{‡} | CDK1, CDK2, CDK5 and CDK9 (NA) |
| SNS-032, also known as BMS- | CDK2, CDK7 and CDK9 (CDK1 and CDK4) |
| 387032 (Sunesis) ^{‡} | |
| Terameprocol, also known as | CDK1, surviving and VEGFRs (NA) |
| EM-1421 (Erimos) ^{‡} | |
| ZK 304709, also known as | CDK1, CDK2, CDK4, CDK7 and CDK9 |
| MTGI and ZK-CDK (Schering AG)* | (VEGFR1 VEGFR2, VEGFR3 and PDGFRβ) |
| Abeciclib (Eli Lily) | CDK4, CDK6 |
| Ribociclib (Chemietek) | CDK4, CDK6 |

Data extracted from http://www.clinicaltrials.gov. ^{‡}Intravenous.*Oral.§ Indisulam is not a direct CDK inhibitor: it causes a depletion of cyclin E levels, which reduces CDK2 activity, and a depletion of cyclin H levels, which reduces CDK7 activity. CDK, cyclin-dependent kinase; CLL, chronic lymphocytic leukaemia; CYC, cyclin; DYRK1A, dual specificity tyrosine-phosphorylation-regulated kinase 1A; ERK, extracellular signal-regulated kinase; G1, first gap; G2, second gap; GSK3beta, glycogen synthase kinase 3beta; IC50, compound concentration that caused 50% inhibition of kinase activity (in vitro kinase assays) or cellular proliferation (cell proliferation assays); Ki, inhibition constant; M, mitosis; NA, not available; NHL, non-Hodgkin's lymphoma; NSCLC, non-small-cell lung carcinoma; PDGFRbeta, platelet-derived growth factor receptor-beta; PDXK, pyridoxal kinase; RB 1, retinoblastoma protein; S, synthesis; VEGFR, vascular endothelial growth factor receptor.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating cancer in a subject, comprising:
a) assessing pY88 phosphorylation levels in p27 in a biological sample comprising cancer cells from a subject and stratifying Y88 phosphorylation levels as 0, 1, 2 or 3 as compared to pY88 phosphorylation levels observed in control tissues;
b) correlating a level of 0 with cd4k inhibitor insensitivity and a level of 1, 2 or 3 with sensitivity to cdk4 inhibition, and
c) administering to subjects identified in step b) as sensitive to cdk4 inhibition, a therapeutically effective amount of at least one cdk4 inhibitor for the alleviation of cancer burden or symptoms.

2. The method of claim 1, wherein said Y88 phosphorylation level is a 1, and said subject is responsive to cdk4 inhibitor therapy.

3. The method of claim 1, wherein said Y88 phosphorylation level is a 2, and said subject is responsive to cdk4 inhibitor therapy.

4. The method of claim 1, wherein said Y88 phosphorylation level is a 3, and said subject is responsive to cdk4 inhibitor therapy.

5. The method of claim 1, wherein said cancer is a cancer is at least one of breast, brain, thyroid, prostate, colorectum, pancreas, cervix, stomach, endometrium, liver, bladder, ovary, testis, head, neck, skin, mesothelial lining, white blood cell, esophagus, muscle, connective tissue, lung, adrenal gland, thyroid, kidney, bone, and stomach.

6. The method of claim 2, wherein said cancer is breast cancer.

7. The method of claim 2, wherein said subject is a human.

8. The method of claim 1, wherein said cdk4 inhibitor is selected from cdk4 inhibitors listed in table 2.

9. The method of claim 1, further comprising administration of a cdk2 inhibitor.

10. The method of claim 1, further comprising administration of an anti-cancer agent.

11. The method of claim 1, wherein said cdk4 inhibitor is an Alt-Brk mimetic.

12. The method of claim 1, wherein said inhibitor is Palbociclib.

13. The method of claim 12, further comprising administration of an Alt-Brk mimetic which acts synergistically with said Palbociclib to kill cancer cells.

14. The method of claim 13, wherein said Alt-brk mimetic which lacks exon 2 and includes the SH3 domain of Brk.

15. A method for assessing efficacy of inhibition of cdk4 activity in cancer treatment comprising; comparing pY88 phosphorylation levels in p27 in biological samples comprising cancer cells from said subject before and after treatment with an anticancer agent, wherein said anti-cancer agent comprises one or more cdk inhibitors samples and stratifying levels as 0 or, 1, 2, or ≥3, wherein a reduction in Y88 phosphorylation level is correlated with efficacy of cdk4 inhibition and reduced cancer cell proliferation and an increase of Y88 is correlated with reduced or loss of efficacy of cdk4 inhibitor therapy.

16. A kit for practicing the method of claim 1, comprising reagents suitable for determining phosphorylation levels of Y88 and or, Y89 in p27, comprising, antibodies which are immunologically specific for detection of phosphorylated and non phosphorylated Y88 and, or Y89, said antibodies comprising a non naturally occurring detectable label, and, or optionally being affixed to a solid support, said kit comprising phosphorylated, and non phosphorylated Y88 and or Y89 antigens.
